(19) United States
(12) Patent Application Publication
(10) Pub. No.: US 2002/0091142 A1
(43) Pub. Date: Jul. 11, 2002

(54) Alpha4beta1 and alpha4beta7 integrin inhibitors

(76) Inventors: Nigel Graham Cooke, Boonton, NJ (US); Michael Lloyd Sabio, Randolph, NJ (US)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 09/803,303

(22) Filed: Mar. 9, 2001

Related U.S. Application Data
(60) Provisional application No. 60/304,184, filed on Mar. 14, 2000.

(51) Int. Cl. C07F 7/10; C07D 213/81; A61K 31/44; A61K 31/425

(52) U.S. Cl. 514/332; 514/370; 514/426; 514/539; 546/309; 548/557; 556/410; 560/38

(57) ABSTRACT

Compounds of the formula I

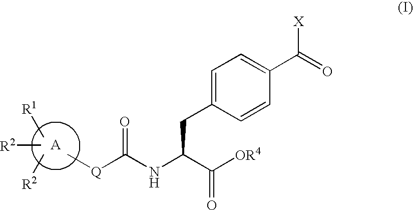

as defined and their pharmaceutically acceptable salts are inhibitors of α₄β₁ and/or α₄β₇ integrins, and are useful in inhibiting cell adhesion and in the therapeutic or prophylactic treatment of transplant rejection and inflammatory and autoimmune diseases.

This application claims benefit of U.S. Provisional Application No. 60/xxx,xxx filed Mar. 14, 2000, which was converted from U.S. application Ser. No. 09/525,700.

The present invention relates to compounds which inhibit α₄-mediated cellular adhesion, and as such are useful in the treatment of various conditions such as acute or chronic organ transplant rejection, inflammatory bowel disease, asthma, diabetes, and rheumatoid arthritis.

Cellular adhesion (i.e. a process by which cells associate with each other, migrate towards a specific target, or localize within the extracellular matrix) underlies many biological phenomena. Cell adhesion causes adhesion of hematopoietic to endothelial cells and the subsequent migration of those hematopoietic cells out of blood vessels and to the site of injury, thus playing a role in mammalian pathologies such as inflammation and immune reactions.

Various cell-surface macromolecules (known as cell adhesion receptors) mediate cell-cell and cell-matrix interactions. For example, the integrins are the key mediators in adhesive interactions between hematopoietic and other cells. Integrins are non-covalent heterodimeric complexes consisting of two subunits, α and β. Depending on the type of its α and β subunit components, each integrin molecule is categorized into its own subfamily. There are at least 12 different α subunits (α1-α6, α-L, α-M, α-X, α-IIB, α-V, and α-E) and at least 9 different β subunits (β1-β9).

The very late antigen-4 (VLA-4), also known as α₄β₁ integrin or CD49d/CD29, is a leukocyte cell surface receptor that participates in a variety of cell-cell and cell-matrix adhesions. It is a receptor for both the cytokine-inducible endothelial cell surface protein, vascular cell adhesion molecule-1 (VCAM-1), and the extracellular matrix protein fibronectin (FN). Anti-VLA-4 monoclonal antibodies (mAb's) inhibit VLA-4-dependent adhesive interactions both in vitro and in vivo. This inhibition of VLA-4-dependent cell adhesion may prevent or inhibit several inflammatory and autoimmune pathologies.

The expression of α₄β₇ integrin on a variety of leukocytes and the increase in α₄β₇ integrin positive cells in diseased tissues indicates that this receptor may also play an important role in cellular recruitment to other sites of inflammation in addition to trafficking to the gut. For example, CD4+, CD8+ T cells, B-cells, NK cells and eosinophils from human peripheral blood were shown to express high levels of α₄β₇ [Picarella et al., J. Immunol. 158:2099-2106 (1997)].

The α₄β₇ integrin complex has three known ligands, VCAM, CS-1, and MAdCAM, of which VCAM and CS-1 are two ligands which are shared by α₄β₁ and α₄β₇.

The use of monoclonal antibodies against integrins such as α₄β₁ and α₄β₇ has demonstrated that a number of integrins are valid therapeutic targets for various inflammatory conditions or diseases.

The present invention provides compounds of formula I

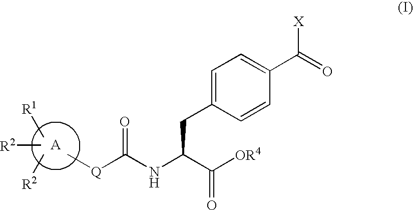

wherein

Ring A is aromatic or heterocyclic,

Q is a direct bond, a carbonyl, lower alkylene optionally substituted by hydroxyl or phenyl, lower alkenylene, or -O-(lower alkylene),

X is OR⁵ or NR⁵R⁶;

R¹, R² and R³ are independently hydrogen, halogen, hydroxyl, alkyl, alkoxy, nitro, amino, carboxyl or an amide or an ester thereof, cyano, alkylcarbonyl, alkylthio, alkylsulfonyl, sulfamoyl, phenyl or heterocyclic, or

two of R¹, R² and R³ together form lower alkylenedioxy,

R⁴ is hydrogen, lower alkyl, lower alkyl interrupted by one or more oxygens, alkenyl, alkynyl, N-morpholinoalkyl, or amino-lower alkyl wherein the nitrogen of the amino group is optionally mono- or di-substituted by lower alkyl,

R⁵ and R⁶ are independently hydrogen, lower alkyl, lower alkyl substituted by fluorine, aryl or heteroaryl each of which substituted by R¹, R² and R³ as defined above, or R⁵ and R⁶ together with the nitrogen atom are a heterocylic ring substituted by R¹, R² and R³ as defined above, with the proviso that when Ring A is thiazolidine wherein the nitrogen is substituted by lower alkyl carbamoyl and X is NR⁵R⁶, then R⁵ and R⁶ are not substituted aryl, and the pharmaceutically acceptable salts thereof.

The present invention also relates to a method for treating or preventing diseases or conditions caused by α₄ (in particular, α₄β₁ and/or α₄β₇)-mediated cell adhesion which comprises administering an effective amount of a compound of the formula I to a subject in need thereof.

Alkyl signifies a straight or branched-chain radical containing from 1 to 10 carbon atoms.

Alkenyl and alkynyl signify a straight or branched-chain radical containing from 2 to 10 carbon atoms.

The use of “lower” preceding the term “alkyl” or “alkoxy” means a said radical containing 1 to 6 carbon atoms. The use of “lower” preceding “alkanoyl,” “alkenyl” or “alkynyl” means a radical containing 2 to 7 carbon atoms.

“Aromatic” means a 5- or 6-membered carbocyclic aromatic ring; a bicyclic 9- or 10-membered aromatic ring, or a tricyclic 13- or 14-membered aromatic ring. Examples of aromatic rings are phenyl, naphthyl, indenyl, indanyl, azulenyl, fluorenyl, anthracenyl.

“Heterocyclic” means a 3-7 membered monocyclic heterocyclic ring or an 8-11 membered bicyclic heterocyclic ring, which is saturated or unsaturated, and which may be optionally benzofused.

Each such heterocycle ring consists of one or more carbon atoms and from one to four heteroatoms selected from nitrogen, oxygen, and sulfur, any oxidized form of nitrogen and sulfur, and the quaternized form of any basic nitrogen. Any ring nitrogen may be optionally substituted by R⁵, as defined herein for compounds of formula I, or by lower alkyl carbamoyl. A heterocycle may be attached at any endocyclic carbon or heteroatom which results in the creation of a stable structure.

Included within the definition of “heterocyclic” are heteroaryl groups such as furyl, thienyl, pyridyl, pyrrolyl, oxazolyly, thiazolyl, imidazolyl, pyrazolyl, 2-pyrazolinyl, pyrazolidinyl, isoxazolyl, isothiazolyl, 1,2,3-oxadiazolyl, 1,2,3-triazolyl, 1,3,4-thiadiazolyl, pyridazinyl, pyrimidinyl, pyrazinyl, 1,3,5-triazinyl, 1,3,5-trithianyl, indolizinyl, indolyl, isoindolyl, 3H-indolyl, indolinyl, benzo[b]furanyl, 2,3-dihydrobenzofuranyl, benzo[b]thiophenyl, 1H-indazolyl, benzimidazolyl, benzthiazolyl, purinyl, 4H-quinolizinyl, quinolinyl, isoquinolinyl, cinnolinyl, phthalazinyl, quinazolinyl, quinoxalinyl, 1,8-naphthyridinyl, pteridinyl, carbazolyl, acridinyl, phenazinyl, phenothiazinyl, and phenoxazinyl.

Also included within the definition of “heterocyclic” are saturated heterocycles such as piperidine, morpholine, pyrrolidine, thiazolidine, and piperazine.

“Halogen” or “halo” means fluoro, chloro, bromo, and iodo.

Some of the compounds described herein contain one or more asymmetric centers and may thus give rise to enantiomers, diastereomers, and other stereoisomeric forms which may be defined in terms of absolute stereochemistry as (R) or (S), or as (D) or (L) for amino acids. Unless otherwise indicated, the present invention is meant to include all such possible stereoisomers as well as their racemic and optically pure forms. Optically active (R) and (S), or (D) and (L), isomers may be prepared using chiral synthons or chiral reagents, or resolved using conventional techniques. When the compounds described herein contain olefinic double bonds or other centers of geometric asymmetry, and unless specified otherwise, it is intended to include both E and Z geometric isomers. Likewise, all tautomeric forms are intended to be included.

In one embodiment, Ring A is aromatic, for example Ring A may be phenyl, especially phenyl wherein R¹, R² and R³ are independently hydrogen, lower alkyl or halo.

More preferably, when Ring A is phenyl, then one of R¹, R² and R³ is hydrogen, and two of R¹, R² and R³ are independently lower alkyl (e.g., methyl) or halo (e.g., bromo or chloro), and are most preferably in the -2 and -6 positions of the phenyl ring, respectively.

In another embodiment, Ring A is a 5- or 6- membered heterocyclic ring having 1 to 3 heteroatoms selected from sulfur, oxygen and nitrogen, said nitrogen being optionally substituted by lower alkylcarbonyl or lower alkylsulfonyl. When Ring A is an unsaturated heterocycle such as pyridyl, then R¹, R² and R³ are preferably independently hydrogen, lower alkyl or halo. When Ring A is a saturated or partially saturated nitrogen-containing ring, such as for example, piperidine, pyrrolidine, oxazolidine or thiazolidine, then R¹, R² and R³ are preferably hydrogen.

Various illustrative embodiments of Ring A including R¹, R² and R³ are as follows

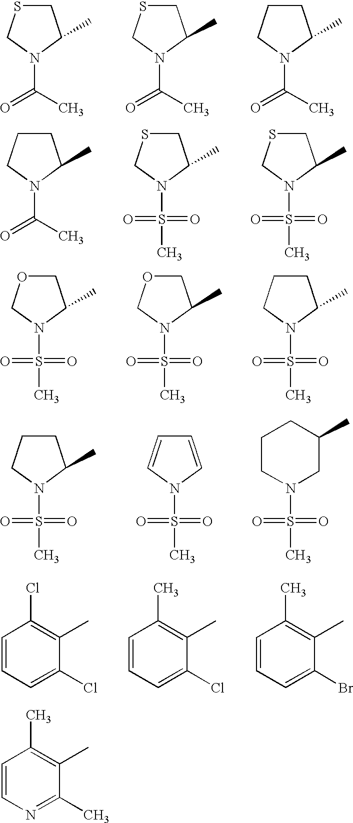

Ring A is even more preferably Ring Aa as follows:

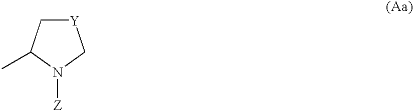

wherein Y is O, S, or (CH₂)ₙ;

n is 1 or 2; and

Z is lower alkylcarbonyl (e.g., acetyl) or lower alkylsulfonyl (e.g., methyl sulfonyl).

A particular example of said Ring Aa is, for example, Ring Aa₁ as follows:

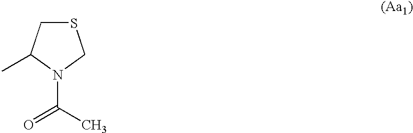

R⁴ is preferably hydrogen, lower alkyl (e.g., C₁-C₄alkyl), lower alkyl interrupted by one or two oxygens, N-morpholinoalkylene or di (C₁-C₂alkyl)amino (C₁-C₂alkyl). R⁴ may be, for example, methyl, ethyl, isopropyl, n-butyl, isopentyl, —CH₂CH₂OCH₃, CH₂CH₂OCH₂CH₂OCH₃, N-morpholino methylene or dimethylaminomethylene.

R⁵ and R⁶ are independently preferably hydrogen or lower alkyl, more preferably C₁-C₄alkyl (e.g., t-butyl or i-propyl).

Preferably, when Ring A is thiazolidine wherein the nitrogen is substituted by lower alkyl carbamoyl and X is NR⁵R⁶, then R⁵ and R⁶ are not substituted aryl and more preferably are neither substituted nor unsubstituted aryl.

In formula I the following significances are preferred independently, collectively or in any combination or sub-combination:

(a) A is phenyl, thiazolidinyl, pyridinyl or pyrrolidinyl;

(b) Q is a direct bond;

(c) X is OR⁵ or NR⁵R⁶;

(d) R¹, R² and R³ are independently hydrogen, halogen, C₁-C₆alkyl, acetyl or C₁-C₄alkylsulfonyl;

(e) R⁴ is hydrogen or C₁-C₄alkyl; and

(f) R⁵ is hydrogen, C₁-C₆alkyl, phenyl substituted by one or more substituents selected from C₁-C₆alkyl, halogen, cyano, or pyridinyl substituted by one or more substituents selected from C₁-C₆alkyl or halogen and R⁶ is hydrogen or C₁-C₆alkyl.

A preferred group are compounds wherein A is phenyl substituted by one or more substituents selected from halogen and C₁-C₄alkyl, thiazolidinyl substituted by acetyl, pyridinyl substituted by C₁-C₄alkyl, or pyrrolidinyl substituted by C₁-C₄alkylsulfonyl; Q is a direct bond; X is OR⁵ or NR⁵R⁶ wherein R⁵ is hydrogen, C₁-C₆alkyl, phenyl substituted by one or more substituents selected from C₁-C₄alkyl, halogen or cyano, or pyridinyl substituted by one or more substituents selected from C₁-C₄alkyl or halogen and R⁶ is hydrogen or C₁-C₄alkyl; and R⁴ is hydrogen or C₁-C₄alkyl.

More preferred are compounds wherein A is 2,6-dichlorophenyl, 2-chloro-6-methylphenyl, 2,4-dimethylpyridin-3-yl, 3-acetyl-thiazolidin-4-yl, or 1-methanesulfonylpyrrolidin-2-yl; Q is a direct bond; X is OH, O-butyl, O-2,6-dichlorophenyl, O-2,6-difluorophenyl, O-2,4,6-trichlorophenyl, O-2-chloro-6-methylphenyl, O-2-cyanophenyl, O-2,6-dimethylphenyl, O-2-bromopyridin-3-yl, O-2-chloropyridin-3-yl, O-2-methylpyridin-3-yl, O-2,6-dimethylpyridin-3-yl, NH-butyl, NH-2-fluorophenyl, NH-2,6-difluorophenyl, NH-2,6-dichlorophenyl, NH-2-chloro-6-methyl-phenyl, NH-2,6-dimethylphenyl, NH-2-cyanophenyl, NH-2-chloro-pyridin-3-yl, N(CH₃)-2-chlorophenyl, N(CH₃)-2,6-dichlorophenyl, or morpholin-4-yl; and R⁴ is hydrogen or methyl.

In addition to the foregoing the present invention also provides a process for the production of a compound of formula I which process comprises coupling an aromatic or heterocyclic moiety to a suitably substituted amino carbonylethyl benzoic acid or benzoate optionally followed by further derivatisation according to methods known to the skilled artisan.

More particularly the invention provides a process for the production of a compound of formula I

which process comprises

reacting a compound of formula II

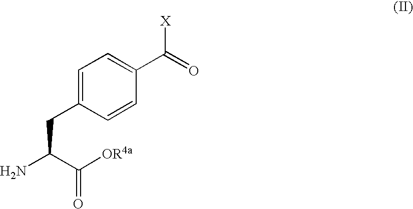

wherein X is as defined above and R^{4a} is R⁴ as defined above or unsubstituted or substituted silanyl alkyl with a compound of formula III

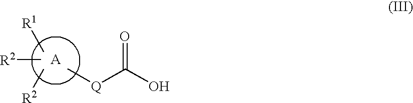

wherein Ring A, Q, R¹, R² and R³ are as defined above, optionally followed by desilylation and further derivatisation.

Compounds of formula II wherein R^{4a} is silanyl alkyl, e.g. 2-diphenylmethyl silanyl ethyl or 2-trimethylsilanyl ethyl, are novel and also an embodiment of the present invention.

Compounds of formula II wherein R^{4a} is R⁴, and compounds of formula III are known or may be prepared using known techniques using known or commercially available starting materials.

A representative reaction scheme for the preparation of compounds of formula I wherein Ring A, Q, X, R¹, R² and R³ are as defined above and R⁴ is lower alkyl, lower alkyl interrupted by one or more oxygens, alkenyl, alkynyl, N-morpholinoalkyl, or amino-lower alkyl wherein the nitrogen of the amino group is optionally mono- or di-substituted by lower alkyl, is depicted below:

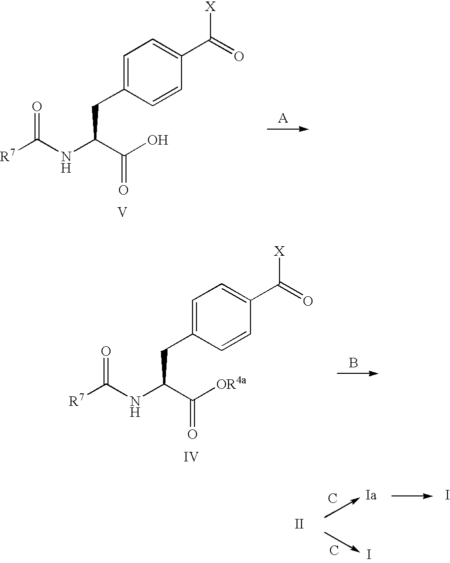

wherein R⁷ is -O-tert-butyl.

| | | typical | typical | typical tempera- |
|---|---|---|---|---|
| step | typical conditions | time | solvents | ture range |
| A | alcohol (R^{4a}OH), 1-[3-(dimethylamino)- | 4 to | DMF, CH₂Cl₂, | −20 to 100° C. |
| | propyl]-3-ethylcarbodiimide hydro- | 24 h | CH₃CN, THF | |
| | chloride, 4-(dimethyl-amino)pyridine, | | | |
| | di-iso-propylethylamine, 1-hydroxy-7- | | | |
| | azabenzotriazole | | | |
| B | hydrogen chloride gas (1 N) in ethyl | 12 to | ethyl acetate | −20 to 70° C. |
| | acetate | 24 h | | |
| C | acid (AQCOOH), 1-[3-(dimethylamino)- | 4 to | DMF, CH₂Cl₂, | −20 to 100° C. |
| | propyl]-3-ethylcarbodiimide hydro- | 24 h | CH₃CN, THF | |
| | chloride, 4-(dimethylamino)pyridine, di- | | | |
| | iso-propylethylamine, 1-hydroxy-7- | | | |
| | azabenzotriazole; or | | | |
| | acid chloride (AQCOCI), di-iso- | 4 to | DMF, THF, | −20 to 100° C. |
| | propylethylamine | 24 h | CH₂Cl₂ | |

Compounds of formula I wherein R⁴ is hydrogen may be prepared by reacting a compound of formula Ia

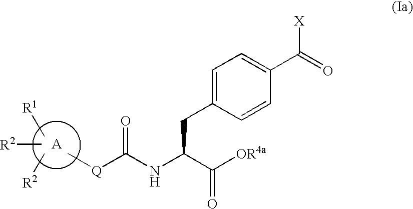

wherein R^{4a} is lower alkyl, lower alkyl interrupted by one or more oxygens, alkenyl, alkynyl, N-morpholinoalkyl, amino-lower alkyl wherein the nitrogen of the amino group is optionally mono- or di-substituted by lower alkyl, or unsubstituted or substituted silanyl alkyl, for 0.5 to 24 h at a temperature in the range of from −20 to 100° C. in the presence of aqueous lithium hydroxide and THF; or for 0.1 to 24 h at a temperature in the range of from −20 to 90° C. in the presence of 1 M tetrabutylammonium fluoride.

Compounds of formula I wherein X is OH may be prepared by reacting a compound of formula I wherein X is OR⁵ wherein R⁵ is lower alkyl, lower alkyl substituted by fluorine, aryl or heteroaryl each of which substituted by R¹, R² and R³ as defined above, or a heterocylic ring substituted by R¹, R² and R³ as defined above, for 1 to 24 h at a temperature in the range of from −20 to 70° C. with 4 N hydrogen chloride gas in dioxane, and dioxane, THF and CH₂Cl₂; or with 4 N trifluoroacetic acid, and THF and CH₂Cl₂. In analogy thereto, a compound of formula Ia wherein X is OR⁵ may be reacted, followed by converting group R^{4a} to group R⁴, R⁴ being hydrogen.

Compounds of formula I wherein X is OH may be converted to compounds of formula I wherein X is OR⁵ wherein R⁵ is lower alkyl, lower alkyl substituted by fluorine, aryl or heteroaryl each of which substituted by R¹, R² and R³ as defined above, or to compounds of formula I wherein X is NR⁵R⁶ wherein R⁵ and R⁶ are independently hydrogen, lower alkyl, lower alkyl substituted by fluorine, aryl or heteroaryl each of which substituted by R¹, R² and R³ as defined above, or R⁵ and R⁶ together with the nitrogen atom are a heterocylic ring substituted by R¹, R² and R³ as defined above, by reaction for 4 to 24 h at a temperature in the range of from −20 to 100° C. in the presence of amine (XH) or alcohol (XH), 1-[3-(dimethylamino)propyl]-3-ethylcarbodiimide hydrochloride, 4-(dimethylamino)pyridine, diisopropylethyl-amine, 1-hydroxy-7-azabenzotriazole, and DMF, CH₂Cl₂, CH₃CN, THF; or for 6 to 36 h at a temperature in the range of from −20 to 90° C. in the presence of amine (XH) or alcohol (XH), polymer supported triphenylphosphine, triethylamine, CBr₄ or CCl₄, di-iso-propylethylamine, 4-(dimethyl-amino)pyridine, and ClCH₂CH₂Cl, CH₂Cl₂ and THF. In analogy thereto, a compound of formula la wherein X is OH may be reacted, followed by converting group R^{4a} to group R⁴, R⁴ being hydrogen.

The pharmaceutical compositions of the present invention comprise a compound of formula I or a pharmaceutically acceptable salt thereof as an active ingredient, and may also contain a pharmaceutically acceptable carrier and, optionally, other therapeutic ingredients. The term “pharmaceutically acceptable salts” refers to salts prepared from pharmaceutically acceptable non-toxic acids or bases including organic and inorganic acids or bases.

When a compound of the present invention is acidic, salts may be prepared from pharmaceutically acceptable non-toxic bases. Salts derived from all stable forms of inorganic bases include aluminum, ammonium, calcium, copper, iron, lithium, magnesium, manganese, potassium, sodium, zinc, etc. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines and basic ion-exchange resins such as arginine, betaine, caffeine, choline, N,N′-dibenzylethylenediamine, diethylamine, 2-diethyl-aminoethanol, 2-dimethylaminoethanol, ethanolamine, ethylenediamine, N-ethylmorpholine, N-ethylpiperidine, glucamine, glucosamine, histidine, isopropylamine, lysine, methylglucosamine, morpholine, piperazine, piperidine, polyamine resins, procaine, purine, theobromine, triethylamine, trimethylamine, tripropylamine, etc.

When a compound of the present invention is basic, salts may be prepared from pharmaceutically acceptable non-toxic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, mandelic, methanesulfonic, mucic, nitric, pamoic, pantothenic, phosphoric, succinic, sulfuric, tartaric, p-toluenesulfonic, etc. Particularly preferred are citric, hydrobromic, maleic, phosphoric, sulfuric, and tartaric acids. Base salts also include ammonium, alkali metal, and alkaline earth metal salts, salts with organic bases, such as dicyclohexylamine salts, and salts with amino acids such as arginine and lysine. Also, basic nitrogen-containing groups can be quaternized with such agents as lower alkyl halides, such as methyl chloride, dialkyl sulfates, such as dimethyl, sulfates, long chain halides such as stearyl chlorides, and aralkyl halides, such as benzyl chlorides.

The compounds of the invention are particularly useful in mammals as VLA-4 antagonists and as inhibitors of VLA-4 associated cell adhesion. Certain of the compounds inhibit both α₄β¹ and α₄β₇ integrins.

The ability of the compounds of formula I to inhibit α₄β₇ and/or α₄β₇ associated cell adhesions makes them useful for treating, ameliorating, or preventing a variety of inflammatory, immune and autoimmune diseases. Preferably the diseases to be treated with the methods of this invention are selected from respiratory disorders (such as asthma), arthritis, psoriasis, transplantation rejection, multiple sclerosis, type I diabetes, and inflammatory bowel disease, stem cell mobilization and engraftment, and sickle cell anemia.

The compounds of formula I are also useful in transplantation surgery; specifically, for the treatment of xenograft and allograft rejection, both chronic and acute, for the induction of tolerance to donor cells, tissues or organs, or in treating ischemia reperfusion/injury.

The compound of formula I may be administered optionally prior to the transplant operation, and at the time of and/or following the transplant operation.

When used for the suppression of organ transplant rejection, a compound of formula I may be administered as the sole active ingredient or together with other drugs in immunomodulating regimens or other anti-inflammatory agents effective in treating acute or chronic rejection. For example, the compounds of formula I may be used in combination with cyclosporins, rapamycins or ascomycins, or their immunosuppressive analogs, e.g. cyclosporin A, cyclosporin G, FK-506, rapamycin, 40-O-(2-hydroxy)ethyl-rapamycin (RAD), etc.; corticosteroids; cyclophosphamide; azathioprene; methotrexate; brequinar; leflunomide; mizoribine; mycophenolic acid; mycophenolate mofetil (MMF); deoxyspergualins (e.g., 15-deoxyspergualine) and analogs, 2-amino-2-[2-(4-octylphenyl)ethyl]propane-1,3-diol hydrochloride, corticosteroids (e.g., methotrexate, prednisolone, methylprednisolone, dexamethasone), or other immunomodulatory compounds (e.g., CTLA4-lg); anti-LFA-1 or anti-lCAM antibodies, or antibodies to leukocyte receptors or their ligands (e.g., antibodies to MHC, CD2, CD3, CD4, CD7, CD25, CD28, B7, CD40, CD45, CD58, CD152 (CTLA-4), or CD 154 (CD40 ligand).

As to the respiratory diseases, the compounds of the invention are useful as agents for the symptomatic or prophylactic treatment of inflammatory airways diseases. Such diseases include asthma of whatever type or genesis including both intrinsic (non-allergic) asthma and, especially, extrinsic (allergic) asthma. They are useful for the treatment of bronchitic asthma, exercise- induced asthma, occupational asthma, asthma induced following bacterial infection and other non-allergic asthmas. Treatment of asthma is also to be understood as embracing treatment of patients of less than 4 or 5 years of age exhibiting wheezing symptoms, particularly at night and diagnosed or diagnosable as “wheezy infants”.

Prophylactic efficacy in the treatment of asthma may be manifested by reduced frequency or reduced severity of symptomatic attack, improvement in lung function or improved airways hypereactivity. It may be further evidenced by reduced requirement for symptomatic therapy, i.e. therapy for, or intended to restrict or abort, symptomatic attack when it occurs, for example for anti-inflammatory therapy using a corticosteroid.

Other inflammatory airways diseases which may be treated with compounds of the invention include pneumoconiosis (an inflammatory, commonly occupational, disease of the lungs occasioned by repeated inhalation of dusts) including for example aluminosis, asbestosis, chalicosis, siderosis, silicosis, tabacosis and byssinosis.

Further inflammatory airways diseases which may be treated with compounds of the invention include adult respiratory distress syndrome (ARDS), chronic obstructive pulmonary disease (COPD), including bronchitis and emphysema, and exacerbation of airways hyperactivity consequent to other drug therapy, e.g. aspirin or b-agonist bronchodilator therapy. Also treatable is sarcoidosis.

In view of their anti-inflammatory activity, particularly in relation to inhibition of eosinophil activation, compounds of the invention are also useful for the treatment of related disorders of the airways, e.g. eosinophilia, hypereosinophilia, eosinophilic pneumonia, parasitic infestation (including tropical eosinophilia), bronchopulmonary aspergillosis, polyarteritis nodosa, eosinophilic granuloma and eosinophil-related disorders affecting the airways caused by drug-reaction.

Compounds of the invention may also be used in the treatment of allergic inflammatory diseases such as allergic rhinitis.

In accordance with the foregoing, the invention includes:

(A) the use of a compound of the invention, i.e. a compound of formula I or a pharmaceutically acceptable salt thereof, as hereinbefore described, for the preparation of a medicament for the treatment of inflammatory, immune or autoimmune diseases, particularly arthritis, transplant rejection or inflammatory airways diseases, especially asthma; and

(B) a method of treating an inflammatory, immune or autoimmune disease, particularly arthritis, transplant rejection or an inflammatory airways disease, especially asthma, which comprises administering to a mammal, particularly a human, in need of such treatment a compound of the invention as hereinbefore described.

The dosage in vitro may range between about 10⁻⁶ and 10⁻¹⁰ molar concentrations, preferably between about 10⁻⁷ and 10⁻⁹ molar concentrations.

The magnitude of the prophylactic or therapeutic dose of the compounds of the invention will vary with the nature and severity of the condition to be treated with the mammal involved and with the particular compound of the invention and its route of administration. In general, the daily dose range lies in the range of 200 to 0.001 mg/kg body weight of a mammal, preferably 50 to 0.05 mg/kg, and most preferably 1.0 to 0.1 mg/kg, in single or divided doses. In some cases, it may be necessary to use doses outside these ranges. When a composition for intravenous administration is employed, a suitable daily dosage range is from about 50 to 0.0005 mg (preferably 20 to 0.01 mg) compound of the invention per kg body weight. When a composition for oral administration is employed, a suitable daily dosage range is from about 20 to 0.001 mg (preferably 10 to 0.01 mg) compound of the invention per kg body weight. When a composition for ophthalmic administration is employed, a suitable daily dosage range is from about 10-0.01% (preferably 5.0-0.5% compound of the invention, typically prepared as a 2.0-0.1% by weight solution or suspension of the compound in an acceptable ophthalmic formulation.

The compounds of the invention may also be used in combination with other pharmaceutically active ingredients.

For example, a typical ocular formulation may comprise the compound alone or in combination with a b-adrenergic blocking agent such as timolol maleate or a parasympathomimetic agent such as pilocarpine. When used in combination, the two active ingredients are present in approximately equal parts.

Any suitable route of administration may be employed or providing a mammal, especially a human, with an effective dosage of a compound of the invention. For example, oral, rectal, topical, parenteral, ocular, pulmonary, nasal, etc. routes may be employed. Dosage forms include tablets, troches, dispersions, suspensions, solutions, capsules, creams, ointments, aerosols, and the like.

The pharmaceutical compositions of the present invention comprise a compound of formula I, or a pharmaceutically acceptable salt thereof, as an active ingredient, and may also contain a pharmaceutically acceptable carrier and, optionally, other therapeutically active ingredients. The invention includes such compositions for use in the treatment of an inflammatory, immune or autoimmune disease, particularly arthritis, transplant rejection or an inflammatory airways disease, especially asthma.

The compositions include compositions suitable for oral, rectal, topical (including transdermal devices, aerosols, creams, ointments, lotions, and dusting powders), parenteral (including subcutaneous, intramuscular, and intravenous), ocular (ophthalmic), pulmonary (nasal or buccal inhalation), or nasal administration; although the most suitable route in any given case will depend largely on the nature and severity of the condition being treated and on the nature of the active ingredient. They may be conveniently presented in unit dosage form and prepared by any of the methods well known in the art of pharmacy.

For example, in the treatment of airways diseases, compounds of the invention may be administered orally, for example in tablet form, or by inhalation, for example in aerosol or other atomisable formulations or in dry powder formulations, using an appropriate inhalation device such as those known in the art. For use in the treatment of allergic rhinitis, the compounds of the invention may also be administered intranasally.

A compound of the invention may be combined as the active ingredient in intimate admixture with a pharmaceutical carrier according to conventional pharmaceutical compounding techniques. The carrier may take a wide variety of forms depending on the nature of the preparation desired for administration, i.e., oral, parenteral, etc. In preparing oral dosage forms, any of the usual pharmaceutical media may be used, such as water, glycols, oils, alcohols, flavoring agents, preservatives, coloring agents, and the like in the case of oral liquid preparations (e.g., suspensions, elixirs, and solutions); or carriers such as starches, sugars, microcrystalline cellulose, diluents, granulating agents, lubricants, binders, disintegrating agents, etc. in the case of oral solid preparations such as powders, capsules, and tablets. Solid oral preparations are preferred over liquid oral preparations. Because of their ease of administration, tablets and capsules are the preferred oral dosage unit form. If desired, capsules may be coated by standard aqueous or non-aqueous techniques.

In addition to the dosage forms described above, the compounds of the invention may be administered by controlled release means and devices.

Pharmaceutical compositions of the present invention suitable for oral administration may be prepared as discrete units such as capsules, cachets, or tablets each containing a predetermined amount of the active ingredient in powder or granular form or as a solution or suspension in an aqueous or nonaqueous liquid or in an oil-in-water or water-in-oil emulsion. Such compositions may be prepared by any of the methods known in the art of pharmacy. In general, the compositions are prepared by uniformly and intimately admixing the active ingredient with liquid carriers, finely divided solid carriers, or both and then, if necessary, shaping the product into the desired form. For example, a tablet may be prepared by compression or molding, optionally with one or more accessory ingredients. Compressed tablets may be prepared by compressing in a suitable machine the active ingredient in a free-flowing form such as powder or granule optionally mixed with a binder, lubricant, inert diluent, or surface active or dispersing agent. Molded tablets may be made by molding in a suitable machine, a mixture of the powdered compound moistened with an inert liquid diluent. Ophthalmic inserts are made from compression molded films which are prepared on a Carver Press by subjecting the powdered mixture of active ingredient and HPC to a compression force of 12,000 lb. (gauge) at 149° C. for 1-4 min. The film is cooled under pressure by having cold water circulate in the platen. The inserts are then individually cut from the film with a rod-shaped punch. Each insert is placed in a vial, which is then placed in a humidity cabinet (88% relative humidity at 30° C.) for 24 days. After removal from the cabinet, the vials are capped and then autoclaved at 121° C. for 0.5 hr.

The compositions containing a compound of this invention may also comprise an additional agent selected from the group consisting of cortiocosteroids, bronchodilators, antiasthmatics (mast cell stabilizers), anti-inflammatories, antirheumatics, immunosuppressants, anti-metabolites, immunonodulators, antipsoriatics, and antidiabetics. Specific compounds include theophylline, sulfasalazine and aminosalicylates (anti-inflammatories); or other drugs as mentioned above in connection with suppression of transplant rejection (e.g., cyclosporin, FK-506, and rapamycin (immunosuppressants); cyclophosphamide and methotrexate (antimetabolites) and the like); and interferons (immunomodulators).

The invention includes a compound of the invention as hereinbefore described in inhalable form and an inhalable medicament comprising such a compound in inhalable form optionally together with a pharmaceutically acceptable carrier in inhalable form.

The inhalable form may be, for example, an atomisable composition such as an aerosol comprising the compound of the invention in solution or dispersion in a propellant or a nebulizable composition comprising a dispersion of the compound of the invention in an aqueous, organic or aqueous/organic medium, or a finely divided particulate form comprising the compound of the invention in finely divided form optionally together with a pharmaceutically acceptable carrier in finely divided form.

An aerosol composition suitable for use as the inhalable form may comprise the compound of the invention in solution or dispersion in a propellant, which may be chosen from any of the propellants known in the art. Suitable such propellants include hydrocarbons such as n-propane, n-butane or isobutane or mixtures of two or more such hydrocarbons, and halogen-substituted hydrocarbons, for example fluorine-substituted methanes, ethanes, propanes, butanes, cyclopropanes or cyclobutanes, particularly 1,1,1,2-tetrafluoroethane (HFA134a) and heptafluoropropane (HFA227), or mixtures of two or more such halogen-substituted hydrocarbons. Where the compound of the invention is present in dispersion in the propellant, i.e. where it is present in particulate form dispersed in the propellant, the aerosol composition may also contain a lubricant and a surfactant, which may be chosen from those lubricants and surfactants known in the art. The aerosol composition may contain up to about 5% by weight, for example 0.002 to 5%, 0.01 to 3%, 0.015 to 2%, 0.1 to 2%, 0.5 to 2% or 0.5 to 1%, by weight of the compound of the invention, based on the weight of the propellant. Where present, the lubricant and surfactant may be in an amount up to 5% and 0.5% respectively by weight of the aerosol composition. The aerosol composition may also contain ethanol as co-solvent in an amount up to 30% by weight of the composition, particularly for administration from a pressurised metered dose inhalation device.

A finely divided particulate form, i.e. a dry powder, suitable for use as the inhalable form may comprise the compound of the invention in finely divided particulate form, optionally together with a finely divided particulate carrier, which may be chosen from materials known as carriers in dry powder inhalation compositions, for example saccharides, including monosaccharides, disaccharides and polysaccharides such as arabinose, glucose, fructose, ribose, mannose, sucrose, lactose, maltose, starches or dextran. As especially preferred carrier is lactose. The dry powder may be in capsules of gelatin or plastic, or in blisters, for use in a dry powder inhalation device, preferably in dosage units of 5 μg to 40 mg of the active ingredient. Alternatively, the dry powder may be contained as a reservoir in a multi-dose dry powder inhalation device.

In the finely divided particulate form, and in the aerosol composition where the compound of the invention is present in particulate form, the compound of the invention may have an average particle diameter of up to about 10 μm, for example 1 to 5 μm. The particle size of the compound of the invention, and that of a solid carrier where present in dry powder compositions, can be reduced to the desired level by conventional methods, for example by grinding in an air-jet mill, ball mill or vibrator mill, microprecipitation, spray-drying, lyophilisation or recrystallisation from supercritical media.

The inhalable medicament may be administered using an inhalation device suitable for the inhalable form, such devices being well known in the art. Accordingly, the invention also provides a pharmaceutical product comprising a compound of the invention in inhalable form as hereinbefore described in association with an inhalation device. In a further aspect, the invention provides an inhalation device containing a compound of the invention in inhalable form as hereinbefore described.

Where the inhalable form is an aerosol composition, the inhalation device may be an aerosol vial provided with a valve adapted to deliver a metered dose, such as 10 to 100 μl, e.g. 25 to 50 μl, of the composition, i.e. a device known as a metered dose inhaler. Suitable such aerosol vials and procedures for containing within them aerosol compositions under pressure are well known to those skilled in the art of inhalation therapy. Where the inhalable form is a nebulizable aqueous, organic or aqueous/organic dispersion, the inhalation device may be a known nebulizer, for example a conventional pneumatic nebulizer such as an airjet nebulizer, or an ultrasonic nebulizer, which may contain, for example, from 1 to 50 ml, commonly 1 to 10 ml, of the dispersion; or a hand-held nebulizer such as an AERx (ex Aradigm, US) or BINEB (Boehringer Ingelheim) nebulizer which allows much smaller nebulized volumes, e.g. 10 to 100 μl, than conventional nebulizers. Where the inhalable form is the finely divided particulate form, the inhalation device may be, for example, a dry powder inhalation device adapted to deliver dry powder from a capsule or blister containing a dosage unit of the dry powder or a multidose dry powder inhalation device adapted to deliver, for example, 25 mg of dry powder per actuation. Suitable such dry powder inhalation devices are well known. The compounds of the invention are antagonists of an α₄ integrin, and in particular are inhibitors of α₄β₁ integrin and/or α₄β₇ integrin.

The activities and specificities of the compounds of this invention may be determined using in vitro and in vivo assays.

The cell adhesion inhibitory activity of these compounds may be measured by determining the concentration of inhibitor required to block the binding of VLA4-expressing cells to fibronectin-, CS1- or VCAM-l-coated plates. In this assay microtiter wells are coated with either fibronectin (containing the CS-1 sequence) or CS-1 or VCAM-l. If CS-1 is used, it must be conjugated to a carrier protein, such as bovine serum albumin, in order to bind to the wells. Once the wells are coated, varying concentrations of the test compound are then added together with appropriately labeled, VLA-4-expressing cells. Alternatively, the test compound may be added first and allowed to incubate with the coated wells prior to the addition of the cells. The cells are allowed to incubate in the wells for at least 30 minutes. Following incubation, the wells are emptied and washed. Inhibition of binding is measured by quantitating the fluorescence or radioactivity bound to the plate for each of the various concentrations of test compound, as well as for controls containing no test compound. VLA-4-expressing cells that may be utilized in this assay include Ramos cells, Jurkat cells, A375 melanoma cells, as well as human peripheral blood lymophocytes (PBLs). The cells used in this assay may be fluorescently or radioactively labeled.

The cell adhesion inhibitory activity of these compounds may also be measured by determining the concentration of inhibitor required to block the binding of α₄β₇-expressing cells to fibronectin-, CS1-, VCAM-1- or MadCAM-1 coated plates. In this assay microtiter wells are coated with either fibronectin (containing the CS-1 sequence) or CS-1 or VCAM-1 MadCAM-1. If CS-1 is used, it must be conjugated to a carrier protein, such as bovine serum albumin, in order to bind to the wells. Once the wells are coated, varying concentrations of the test compound are then added together with appropriately labeled, α₄β₇-expressing cells. Alternatively, the test compound may be added first and allowed to incubate with the coated wells prior to the addition of the cells. The cells are allowed to incubate in the wells for at least 30 minutes. Following incubation, the wells are emptied and washed. Inhibition of binding is measured by quantitating the fluorescence or radioactivity bound to the plate for each of the various concentrations of test compound, as well as for controls containing no test compound. α₄β₇-expressing cells that may be utilized in this assay include RPMI-8866 cells, α₄β₇ transfected cells as well as other cells expressing α₄β₇ but not α₄β₁. The cells used in this assay may be fluorescently or radioactively labeled.

A direct binding assay may also be employed to quantitate the inhibitory activity of the compounds of this invention. In this assay, a VCAM-lgG fusion protein containing the first two immunoglobin domains of VCAM (D1D2) attached above the hinge region of an IgG1 molecule (VCAM 2D-lgG), is conjugated to a marker enzyme, such as alkaline phosphatase (AP). The synthesis of this VCAM-lgG fusion is described in WO 90/13300. The conjugation of that fusion to a marker enzyme is achieved by well known crosslinking methods. The VCAM-lgG enzyme conjugate is then placed in the wells of a multi-well filtration plate, such as that contained in the Millipore Multiscreen Assay System (Millipore Corp., Bedford, Mass.). Varying concentrations of the test inhibitory compound are then added to the wells followed by addition of VLA-4-expressing cells. The cells, compound and VCAM-lgG enzyme conjugate are mixed together and allowed to incubate at RT (room temperature). Following incubation, the wells are vacuum drained, leaving behind the cells and any bound VCAM. Quantitation of bound VCAM is determined by adding an appropriate calorimetric substrate for the enzyme conjugated to VCAM-lgG and determining the amount of reaction cell adhesion inhibitory activity.

One or more compounds of the Examples have measured IC₅₀ values for VLA-4 binding of an order as low as 1 nanomolar.

In order to assess the α₄integrin inhibitory specificity of the compounds of this invention, assays for other major groups of integrins, i.e., β2 and β3, as well as other β1 integrins, such as VLA-5 and VLA-6 are performed. These assays may be similar to the adhesion inhibition and direct binding assays described above, substituting the appropriate integrin-expressing cell and corresponding ligand. For example, polymorphonuclear cells (PMNs) express β2 integrins on their surface and bind to ICAM. β3 integrins are involved in platelet aggregation and inhibition may be measured in a standard platelet aggregation assay. VLA-5 binds specifically to Arg-Gly-Asp sequences, while VLA-6 binds to laminin. Compounds of the Examples are found to be selective for α₄β₁ and/or α₄β₇ versus related integrins.

An in vivo assay which tests the inhibition of contact hyper-sensitivity in an animal is described in Chisholm et al., Eur. J. Immunol., 23:682-688 (1993). An assay which measures the inhibition of Ascaris antigen-induced late phase airway responses and airway hyperresponsiveness in asthmatic sheep is described in Abraham et al., J. Clin. Invest., 93:776-87 (1994).

The compounds of the invention may also be tested in the following assay.

Antigen-induced pulmonary eosinophilia in the mouse

Sensitization of mice

Male B6D2F1/J mice are sensitized by i.p. injection of 0.5 mL alum-precipitated antigen containing 8 μg of ovalbumin (OVA) adsorbed to 2 mg of aluminum hydroxide gel in a saline vehicle. Five days later the mice are given a booster injection with OVA/alum. Control animals are sensitized with alum only. Ten mice are used for each group.

Challenge and drug administration

Mice are placed in a 12×14×10 inch plexiglass chamber and exposed to aerosolized OVA (0.5% in saline) for 1 hour at the beginning of the experiment (t=0), and five hours later. Low molecular weight antagonists are dissolved in 2% DMSO and 150 mM TRIS, pH 8.8. A solvent control is included for each experiment. Drugs are administered orally 30 min prior to OVA exposure, and 6 hours after the first OVA exposure.

BAL fluid collection and analysis

Animals are sacrificed by CO₂ asphyxiation 24 hour after the first antigen challenge. The tracheas are exposed and cannulated. The lungs are lavaged with 0.6 mL buffer (Hanks buffered saline with 10 mM Hepes, 0.5% BSA and 10 U/mL heparin). The number of eosinophils in the lavage is assessed by counting the total number of leukocytes and the percentage of eosinophils for each sample.

The % inhibition is calculated by the formula:

1−(# Eos with drug in OA goup−# Eos in no OA goup)×100% (#Eos in OA group−# Eos in no OA group)

where: Eos=average number of eosinophils, OA =challenged and no OA=unchallenged mice.

The invention is further defined by reference to the following examples, which are intended to be illustrative and not limitative of the invention.

Abbrevations: DMF: dimethyl formamide; RT: room temperature; TFA: trifluoroacetic acid; THF: tetrahydrofuran; tic: thin layer chromatography

### EXAMPLE A1

### tert-Butyl 4-((S)-2-amino-2-methoxycarbonyl-ethyl)-benzoate hydrochloride salt (Compound B1)

(a) A mixture of tert-butyl 4-((S)-2-tert-butoxycarbonylamino-2-carboxy-ethyl)-benzoate (0.700 g), 1-[3-(dimethylamino)propyl]-3-ethylcarbodiimide hydrochloride (0.440 g), 4-di-methylamino) pyridine (0.024 g), di-iso-propylethylamine (0.40 ml) in methanol (10 ml) is stirred for 18 hours at RT. After checking completion of the reaction by tic, the mixture is evaporated under reduced pressure. The residue is dissolved in ethyl acetate and the organic phase is washed successively with 0.5 N aqueous hydrochloric acid, 0.5 N aqueous sodium bicarbonate solution and brine. The organic layers are dried over magnesium sulphate, filtered and evaporated under reduced pressure to afford an oil which is purified by Biotage flash chromatography using 17% ethyl acetate in hexanes as eluant to afford tert-butyl 4-((S)-2-tert-butoxycarbonylamino-2-methoxycarbonyl-ethyl)-benzoate.

(b) A solution of the compound prepared in (a) (0.322 g) in 1 N hydrogen chloride in ethyl acetate (20 ml) is stirred at RT for 6 hours. The mixture is evaporated under reduced pressure and the resulting white solid dried under high vacuum to afford Compound B1. Mass spec. (ES): (m+H)⁺m/z=280.

### EXAMPLE A2

### tert-Butyl 4-((S)-2-amino-2-[2-(diphenylmethylsilanyl)-ethoxycarbonyl]-ethyl)-benzoate hydrochoride salt (Compound B2)

A mixture of tert-butyl 4-((S)-2-tert-butoxycarbonylamino-2-carboxy-ethyl)-benzoate (9.1 mmol), (2-hydroxyethyl)dipheny-methylsilane (10.9 mmol), 1-[3-(dimethylamino)propyl]-3-ethyl-carbodiimide hydrochloride (12.0 mmol), 4-(dimethylamino)pyridine (0.46 mmol), di-iso-propylethylamine (12.0 mmol) and 1-hydroxy-7-azabenzotriazole (11.0 mmol) in dry DMF (30 ml) is stirred for 18 hours at RT. After checking completion of the reaction by tic, the mixture is evaporated under reduced pressure. The residue is dissolved in ethyl acetate and the organic phase is washed successively with 0.5 N aqueous hydrochloric acid, 0.5 N aqueous sodium bicarbonate solution and brine. The organic layers are dried over magnesium sulphate, filtered and evaporated under reduced pressure to afford an oil which is purified by Biotage flash chromatography using 20% ethyl acetate in hexanes to afford tert-butyl 4-((S)-2-tert-butoxycarbonylamino-2-[2-(diphenylmethyl-silanyl)-ethoxycarbonyl]-ethyl)-benzoate which, using the procedure described in Example A1 (b), affords compound B2 Mass spec. (ES): (m+H)⁺m/z=490.

### EXAMPLE A3

### tert-Butyl 4-((S)-2-amino-2-(2-trimethylsilanylethoxycarbonyl)-ethyl)-benzoate hydrochloride salt (Compound B3)

Using the procedure described for the preparation of compound B2, tert-butyl 4-((S)-2-tert-butoxycarbonylamino-2-carboxyethyl)-benzoate and 2-trimethylsilanylethanol afford compound B3 Mass spec. (ES): (m+H)⁺m/z=366.

### EXAMPLE A4

### (S)-1-Methanesulfonyl-pyrrolidine-2-carboxylic acid (Compound C2)

(a) A solution of tert-butyl (S)-pyrrolidine-2-carboxylate (14.6 mmol) in pyridine (10 ml) is cooled to 0° C. under an inert atmosphere. Methane sulphonyl chloride (15.2 mmol) is added and the mixture is stirred for 1 hour at 0° C. and then for a further 18 hours at RT. The mixture is filtered and the filtrate is evaporated under reduced pressure. The residue is dissolved in ethyl acetate and the ethyl acetate phase is washed with 1 N aqueous hydrochloric acid and water. The organic phase is dried over magnesium sulphate and evaporated to dryness to afford tert-butyl (S)-1-methanesulfonyl-pyrrolidine-2-carboxylate (Mass spec. (ES): (m+NH₄)⁺m/z=267). (b) A solution of tert-butyl (S)-1-methanesulfonyl-pyrrolidine-2-carboxylate (2.01 mmol) in TFA (3 ml) is stirred at RT for 30 min. The mixture is evaporated under reduced pressure and the residue is washed repeatedly with ether followed by hexane and then dried under high vacuum to afford compound C2 (Mass spec. (ES⁻) (m−H)⁺m/z=192).

### EXAMPLE 1

### tert-Butyl 4-{(S)-2-[((S)-3-acetyl-thiazolidine-4-carbonyl)-amino]-2-meth-oxycarbonyl-ethyl}-benzoate (Compound 1)

A mixture of compound B1 (1.1 g), (S)-3-acetyl-thiazolidine-4-carboxylic acid (0.67 g), 1-[3-(dimethylamino)propyl]-3-ethylcarbodiimide hydrochloride (0.87 g), 1-hydroxy-7-aza-benzotriazole (0.57 g), di-iso-propylethylamine (0.6 ml) in DMF (30 ml) is stirred for 18 hours at RT. After checking completion of the reaction by tic, the mixture is evaporated under reduced pressure. The residue is dissolved in ethyl acetate and the organic phase is washed with brine. The organic layers are dried over sodium sulphate, filtered and evaporated under reduced pressure. The residue is purified by Biotage flash chromatography using ethyl acetate as eluant to afford compound 1. Mass spec (ES): (m+1)⁺m/z=437.

### EXAMPLE 2

### tert-Butyl 4-{(S)-2-[((S)-3-acetyl-thiazolidine-4-carbonyl)-amino]-2-carboxy-ethyl}-benzoate (Compound 2)

Lithium hydroxide (0.008 g) is added to a solution of compound 1 in THF (3 ml) and water (1 ml) and the resulting mixture is stirred for 2 hours at RT. Saturated ammonium chloride solution is added and the THF is removed by evaporation under reduced pressure. The resulting aqueous solution is adjusted to pH 2 by the addition of citric acid and then repeatedly extracted with ethyl acetate. The organic phase is dried over sodium sulphate, filtered and evaporated. The residue is purified by preparative hplc on a C18 column using acetonitrile and water containing 0.5% TFA as eluant to afford compound 2. Found: C, 55.02; H, 5.75; N, 5.98%. C₂₀H₂₆N₂O₆S. 0.2 CF₃CO₂H requires C, 55.02; H, 5.93; N, 6.29%.

### EXAMPLE 3

### 4-{((S)-2-[((S)-3-Acetyl-thiazolidine4-carbonyl)-amino]-2-methoxycarbonyl-ethyl}-benzoic acid (Compound 3)

A solution of Compound 1 (1.07 g) in 4 N hydrogen chloride in dioxane, is stirred for 18 hours at RT. The resulting mixture is evaporated under reduced pressure to afford compound 3. Mass spec (ES): (m+Na)⁺m/z=403.

### EXAMPLE 4

### Methyl (2S)-2-[((S)-3-acetyl-thiazolidine-4-carbonyl)-amino]-3-(4-tert-butyl-carbamoyl-phenyl)-propionate (Compound 4)

A mixture of Compound 3 (0.131 mmol), polymer supported triphenylphosphine (0.13 g of 3 mmol/g resin) and triethylamine (0.39 mmol) in dichloromethane (4 ml) is stirred at RT under nitrogen. A solution of carbon tetrabromide (0.048 g, 0.145 mmol) in dichloromethane (2 ml) is added and the resulting mixture is stirred for 2 hours at RT. tert-butylamine (0.26 mmol) is added and the reaction is stirred for a further 18 hours at RT. The reaction mixture is filtered and basic ion exchange resin is added to the filtrate, the mixture is stirred for 5 min and then filtered. The filtrate is evaporated under reduced pressure and the product purified by Biotage flash chromatography using ethyl acetate as eluant to afford a viscous oil. Purification by preparative hplc on a C18 column using acetonitrile and water containing 0.5% TFA as eluant followed by freeze drying of the product containing fractions affords compound 4. Mass spec (ES): (M+H)⁺m/z=436.

Using the procedure described in Example 2 compound 4 affords (2S)-2-[((S)-3-acetyl-thiazolidine-4-carbonyl)-amino]-3-(4-tert-butylcarbamoyl-phenyl)-propionic acid (Compound 8). Mass spec. (ES): (m+H)⁺m/z=422.

### EXAMPLE 5

### Methyl (2S)-2-[(S)-3-acetyl-thiazolidine-4-carbonyl)-amino]-3-[4-(2-chloro-6-methyl-phenylcarbomoyl)-phenyl]-propionate (Compound 5)

A suspension of polymer supported triphenylphosphine (0.13 g of 3 mmol/g resin) in carbon tetrachloride (10 ml) is stirred under reflux for 30 min. The mixture is cooled to RT and a solution of Compound 3 (0.131 mmol) in carbon tetrachloride (2 ml) is added and the mixture is heated under reflux for a further 2 hours. The mixture is cooled to RT and 2 chloro-6-methylaniline (0.26 mmol) is added. The mixture is heated under reflux for a further 18 hours, cooled to RT and filtered. The filtrate is evaporated under reduced pressure and the product is purified by Biotage flash chromatography using a gradient of 50 to 100 % ethyl acetate in hexanes to afford compound 5. Mass spec (ES): (m+Na)⁺m/z=526 and 528. Using the procedure described in Example 2 compound 5 affords (2S)-2-[((S)-3-acetyl-thiazolidine-4-carbonyl)-amino]-3-[4-(2-chloro-6-methyl-phenylcarbamoyl)-phenyl]-propionic acid (Compound 9). Mass spec. (ES): (m+H)⁺m/z=490.

### EXAMPLE 6

### 2,6-Dichlorophenyl 4-((2S)-2-[((S)-3-acetyl-thiazolidine-4-carbonyl)-amino]-2-methoxycarbonyl-ethyl)-benzoate (Compound 6)

A suspension of Compound 3 (0.131 mmol), polymer supported triphenylphoshine (0.13 g, 3 mmol/g resin) and carbon tetrabromide (0.39 mmol) in dry 1,2-dichloroethane (6 ml) is heated under reflux for 6 hours under a nitrogen atmosphere. The mixture is cooled to RT and a solution of 2,6-dichlorophenol (0.26 mmol), di-iso-propylethylamine (0.131 mmol) and 4-(dimethylamino) pyridine (0.007 mmol) in 1,2-dichloroethane (2 ml) is added. The mixture is heated under reflux for 18 hours and then cooled to RT. The mixture is filtered and the filtrate is evaporated under reduced pressure. The residue is purified by Biotage flash chromatography using a gradient of 50 to 70% ethyl acetate in hexanes to afford compound 6. Mass spec (ES): (m+Na)⁺m/z=547 and 549.

Using the procedure described above, the reaction of Compound 3 with 2,4,6-trichlorophenol affords 2,4,6-trichlorophenyl 4-((2S)-2-[((S)-3-acetyl-thiazolidine-4-carbonyl)-amino]-2-methoxycarbonyl-ethyl)-benzoate (Compound 7). Mass spec. (ES): (m+H)⁺m/z=559 and 561. Using the procedure described in Example 2 compound 6 affords 2,6-dichloro-phenyl 4-((2S)-2-[((S)-3-acetyl-thiazolidine-4-carbonyl)-amino]-2-carboxy-ethyl)-benzoate (Compound 10). Mass spec. (ES): (m+H)⁺m/z=511 and 513; and Compound 7 affords 2,4,6-trichlorophenyl 4-((2S)-2-[((S)-3-acetyl-thiazolidine-4-carbonyl)-amino]-2-carboxy-ethyl)-benzoate (Compound 11). Mass spec. (ES): (m+H)⁺m/z=545, 547 and 549.

### EXAMPLE 7

### tert-Butyl 4-[(S)-2-(2,6-dichlorobenzoylamino)-2-(2-trimethylsilanyl-ethoxycarbonyl)-ethyl]-benzoate (Compound 12)

A solution of compound B3 (6.0 mmol) and di-iso-propyl-ethylamine (12.0 mmol) in dichloro- methane (90 ml) is stirred under nitrogen at 0° C. A solution of 2,6-dichlorobenzoyl chloride (7.2 mmol) in dichloromethane (10 ml) is added and the mixture is stirred at RT for 2 hours. The solvent is evaporated under reduced pressure and the residue is purified by Biotage flash chromatography using a gradient of 15 to 20% ethyl acetate in hexanes to afford compound 12. Mass spec. (ES) (m+NH₄)⁺m/z=555 and 557.

### EXAMPLE 8

### tert-Butyl 4-[((S)-2-carboxy-2-(2,6-dichlorobenzoylamino)-ethyl]-benzoate (Compound 13)

A solution of 1 M tetrabutylammonium fluoride in THF (0.3 mmol) is added to a stirred solution of Compound 12 (0.09 mmol) in THF (2 ml). The mixture is stirred at RT for 30 min. The mixture is evaporated under reduced pressure and the residue is purified by preparative hpic on a C18 column using acetonitrile and water containing 0.5% TFA as eluant followed by freeze drying of the product containing fractions to afford compound 13. Mass spec (ES): (m+NH₄)⁺m/z=455 and 457.

Examples 39-40 (Compounds 39 and 40)

Using the procedure described in Example 7, compounds of formula 1 wherein Q is a direct bond, X is O-butyl and R⁴ is hydrogen are prepared by reacting Compound B2 with the corresponding acid chloride in DMF and deprotecting the resulting amide using the procedure described in Example 8.

| | A | Mass spec (ES): m/z |
|---|---|---|
| 39 | 2,4-dimethylpyridin-3-yl | (m + H)⁺: 399 |
| 40 | 2-chloro-6-methyl-phenyl | (m + H)⁺: 418, 420 |

Using the procedures described in Example 1 and in Example 8, compound B2 is reacted with Compound C2 to afford tert-butyl 4-{(S)-2-[((S)-1-methanesulfonyl-pyrrolidine-2-carbonyl)-amino]-2-(2-(diphenylmethylsilanyl)-ethoxycarbonyl]-ethyl}-benzoate which then affords tert-butyl 4-{(2S)-2-carboxy-2-[((2S)-1-methanesulfonyl-pyrrolidine-2-carbonyl)-amino]-ethyl}-benzoate (compound 41). Mass spec. (ES⁻): (m−H)⁺m/z=439.

### EXAMPLE 9

### 2,6-Dichlorophenyl 4-[(S)-2-(2,6-dichlorobenzoylamino)-2-(2-trimethylsilanyl-ethoxycarbonyl)-ethyl]-benzoate (Compound 14)

(a) A solution of Compound 12 (0.072 mmol) in 4 N HCl in dioxane (4 ml) is stirred for 18 hours at RT. The mixture is evaporated under reduced pressure and the residue is purified by preparative hplc on a C18 column using acetonitrile and water containing 0.5% TFA as eluant followed by freeze drying of the product containing fractions to afford 4-[(S)-2-(2,6-dichlorobenzoylamino)-2-(2-trimethylsilanyl-ethoxycarbonyl)-ethyl]-benzoic acid.

(b) A suspension of polymer supported triphenylphoshine (0.062 g, 3 mmol/g resin) and carbon tetrabromide (0.18 mmol) in dry 1,2-dichloroethane (3 ml) is heated under reflux for 0.5 hours under a nitrogen atmosphere. The mixture is cooled to RT and a solution of the compound prepared in (a) (0.06 mmol) in 1,2-dichloroethane (1 ml) is added. The mixture is heated under reflux for 5 hours. The reaction is cooled to RT and a solution of 2,6-dichlorophenol (0.12 mmol), and 4-(dimethylamino)pyridine (0.06 mmol) in 1,2-dichloroethane (1 ml) is added. The mixture is heated under reflux for 18 hours and then cooled to RT. The mixture is filtered and the filtrate is evaporated under reduced pressure. The residue is purified by Biotage flash chromatography using 20% ethyl acetate in hexanes to compound 14. Mass spec (ES): (m+NH₄)⁺m/z=643 and 645.

Using the procedure described in Example 8, compound 14 affords 2,6-dichlorophenyl 4-[(S)-2-carboxy-2-(2,6-dichlorobenzoylamino)-ethyl]-benzoate (Compound 16). Mass spec. (ES): (m+H)⁺m/z=526, 528.

Using the procedure described in Example 14(b), compounds of formula I wherein A is 2,6-dichlorophenyl, Q is a direct bond and R⁴ is hydrogen are prepared by reacting Compound 17 with the corresponding substituted amine or substituted alcohol/phenol and deprotecting the resulting amide or ester using the procedure described in Example 13.

| | | Mass spec (ES): |
|---|---|---|
| | X | m/z &/or Melting point |
| 19 | morpholin-4-yl | (m + H)⁺: 451, 453/61-66° C. |
| 20 | N(CH₃)-2,6-dichlorophenyl | (m + H)⁺: 539, 541/96-100° C. |
| 21 | NH-2-chloro-6-methyl-phenyl | (m + H)⁺: 505, 507/139-142° C. |
| 22 | N(CH₃)-2-chlorophenyl | (m + H)⁺: 505, 507/95-100° C. |
| 23 | NH-2-cyanophenyl | (m + H)⁺: 482, 484/112-115° C. |
| 24 | NH-2,6-dimethylphenyl | (m + H)⁺: 485, 487/138-141° C. |
| 25 | NH-2-chloro-pyridin-3-yl | (m + H)⁺: 492, 494/108-110° C. |
| 26 | NH-2,6-difluorophenyl | (m + H)⁺: 493, 495/125-128° C. |
| 27 | NH-2-fluorophenyl | (m + NH₄)⁺: 492, 494/126-130° C. |
| 28 | NH-2,6-dichlorophenyl | /124-128° C. |
| 29 | NH-butyl | (m + NH₄)⁺: 454, 456/130-133° C. |
| 30 | O-2-cyanophenyl | (m + H)⁺: 483, 485 |
| 31 | O-2,6-difluorophenyl | (m + H)⁺: 494, 496/195-198° C. |
| 32 | O-2-methylpyridin-3-yl | (m + H)⁺: 473, 475/75-80° C. |
| 33 | O-2,6-dimethylpyridin-3-yl | (m + H)⁺: 487, 489/90-94° C. |
| 34 | O-2-chloro-6-methyl-phenyl | (m + H)⁺: 506, 508/120-125° C. |
| 35 | O-2-bromopyridin-3-yl | m⁺: 537, 539, and 541/105-108° C. |
| 36 | O-2,4,6-trichlorophenyl | (m + NH₄)⁺: 577, 579/117-120° C. |
| 37 | O-2,6-dimethylphenyl | (m + H)⁺: 486, 488/117-120° C. |
| 38 | O-2-chloropyridin-3-yl | (m + H)⁺: 493, 495/110-115° C. |

### EXAMPLE 10

### 4-[(S)-2-(2,6-Dichlorobenzoylamino)-2-(2-(diphenylmethylsilanyl)-ethoxy-carbonyl)-ethyl]-benzoic acid (Compound 17)

(a) Using the procedure described in Example 7, Compound B2 is reacted with 2,6-di-chlorobenzoyl chloride to afford tert-butyl 4-[(S)-2-(2,6-dichlorobenzoylamino)-2-(2-(diphenylmethyl-silanyl)-ethoxycarbonyl)-ethyl]-benzoate.

(b) The compound prepared in (a) (4.89 mmol) is dissolved in a solution of 4 N TFA in dichloromethane (50 ml) and the mixture is stirred at RT for 30 min. The mixture is evaporated under reduced pressure and the residue is dissolved in acetonitrile: water (1:1) and freeze dried to afford compound 17. Mass spec. (ES) (m+Na)⁺m/z=628 and 630, (m+H)⁺m/z=606 and 608.

What is claimed is:

1. A compound of the formula I 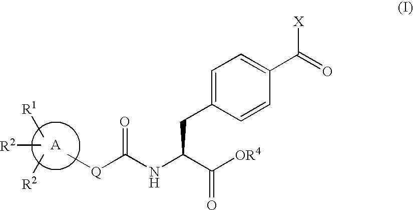
wherein
Ring A is aromatic or heterocyclic,
Q is a direct bond, a carbonyl, lower alkylene optionally substituted by hydroxyl or phenyl, lower alkenylene, or —O—(lower alkylene),
X is OR⁵ or NR⁵R⁶;
R¹, R² and R³ are independently hydrogen, halogen, hydroxyl, alkyl, alkoxy, nitro, amino, carboxyl or an amide or an ester thereof, cyano, alkylcarbonyl, alkylthio, alkylsulfonyl, sulfamoyl, phenyl or heterocyclic, or
two of R¹, R² and R³ together form lower alkylenedioxy,
R⁴ is hydrogen, lower alkyl, lower alkyl interrupted by one or more oxygens, alkenyl, alkynyl, N-morpholinoalkyl, or amino-lower alkyl wherein the nitrogen of the amino group is optionally mono- or di-substituted by lower alkyl,
R⁵ and R⁶ are independently hydrogen, lower alkyl, lower alkyl substituted by fluorine, aryl or heteroaryl each of which substituted by R¹, R² and R³ as defined above, or R⁵ and R⁶ together with the nitrogen atom are a heterocylic ring substituted by R¹, R² and R³ as defined above, with the proviso that when Ring A is thiazolidine wherein the nitrogen is substituted by lower alkyl carbamoyl and X is NR⁵R⁶, then R⁵ and R⁶ are not substituted aryl, and the pharmaceutically acceptable salts thereof.

2. The compound according to claim 1 wherein Q is a direct bond, Ring A is a 5- or 6- membered heterocyclic ring having 1 to 3 heteroatoms selected from sulfur, oxygen and nitrogen, said nitrogen being optionally substituted by lower alkylcarbonyl or lower alkylsulfonyl, and X is OR⁵.

3. The compound according to claim 1 wherein
A is phenyl, thiazolidinyl, pyridinyl or pyrrolidinyl;
Q is a direct bond;
X is OR⁵ or NR⁵R⁶;
R¹, R² and R³ are independently hydrogen, halogen, C₁-C₆alkyl, acetyl or C₁-C₄alkylsulfonyl;
R⁴ is hydrogen or C₁-C₄alkyl; and
R⁵ is hydrogen, C₁-C₆alkyl, phenyl substituted by one or more substituents selected from C₁-C₆alkyl, halogen, cyano, or pyridinyl substituted by one or more substituents selected from C₁-C₆alkyl or halogen and R⁶ is hydrogen or C₁-C₆alkyl.

4. The compound according to claim 1 wherein A is phenyl substituted by one or more substituents selected from halogen and C₁-C₄alkyl, thiazolidinyl substituted by acetyl, pyridinyl substituted by C₁-C₄alkyl, or pyrrolidinyl substituted by C₁-C₄alkylsulfonyl; Q is a direct bond; X is OR⁵ or NR⁵R⁶ wherein R⁵ is hydrogen, C₁-C₆alkyl, phenyl substituted by one or more substituents selected from C₁-C₄alkyl, halogen or cyano, or pyridinyl substituted by one or more substituents selected from C₁-C₄alkyl or halogen and R⁶ is hydrogen or C₁-C₄alkyl; and R⁴ is hydrogen or C₁-C₄alkyl.

5. The compound according to claim 1 wherein A is 2,6-dichlorophenyl, 2-chloro-6-methylphenyl, 2,4-dimethylpyridin-3-yl, 3-acetyl-thiazolidin-4-yl, or 1-methanesulfonylpyrrolidin-2-yl; Q is a direct bond; X is OH, O-butyl, O-2,6-dichlorophenyl, O-2,6-difluorophenyl, O-2,4,6-trichlorophenyl, O-2-chloro-6-methylphenyl, O-2-cyanophenyl, O-2,6-dimethylphenyl, O-2-bromopyridin-3-yl, O-2-chloropyridin-3-yl, O-2-methylpyridin-3-yl, O-2,6-dimethylpyridin-3-yl, NH-butyl, NH-2-fluorophenyl, NH-2,6-difluorophenyl, NH-2,6-dichlorophenyl, NH-2-chloro-6-methyl-phenyl, NH-2,6-dimethylphenyl, NH-2-cyanophenyl, NH-2-chloro-pyridin-3-yl, N(CH₃)-2-chlorophenyl, N(CH₃)-2,6-dichlorophenyl, or morpholin-4-yl; and R⁴ is hydrogen or methyl.

6. A compound of formula II 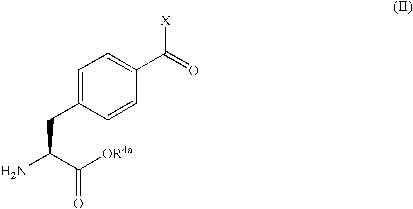
wherein
X is OR⁵ or NR⁵R⁶ wherein R⁵ and R⁶ are independently hydrogen, lower alkyl, lower alkyl substituted by fluorine, aryl or heteroaryl each of which substituted by R¹, R² and R³, or R⁵ and R⁶ together with the nitrogen atom are a heterocylic ring substituted by R¹, R² and R³, wherein R¹, R² and R³ are independently hydrogen, halogen, hydroxyl, alkyl, alkoxy, nitro, amino, carboxyl or an amide or an ester thereof, cyano, alkylcarbonyl, alkylthio, alkylsulfonyl, sulfamoyl, phenyl or heterocyclic, or two of R¹, R² and R³ together form lower alkylenedioxy, and R^{4a} is unsubstituted or substituted silanyl alkyl.

7. A pharmaceutical composition comprising a compound of formula I according to claim 1 and a pharmaceutically acceptable carrier or diluent.

8. A method of treating a disease or condition where α₄β₁ and/or α₄β₇ associated cell adhesion plays a role or is implicated comprising administering to a patient in need thereof an effective amount of a compound according to formula I according to claim 1.

9. A compound according to claim 1 for use in the treatment of the human or animal body.

10. Use of a compound according to claim 1 for the manufacture of a medicament for the treatment or prevention of a disease or condition in which α₄β₁ and/or α₄β₇ integrin inhibition plays a role or is implicated.

11. A compound of formula I according to claim 1 in combination with other pharmaceutically active ingredients.
